(19) United States
(12) Patent Application Publication
(10) Pub. No.: US 2008/0135062 A1
(43) Pub. Date: Jun. 12, 2008

(54) DISINFECTING TABLET

(75) Inventor: Lou D. Hibbard, Eagan, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/609,750

(22) Filed: Dec. 12, 2006

(51) Int. Cl. A61L 2/235

(52) U.S. Cl. 134/7; 510/446

(57) ABSTRACT

A disinfecting tablet is disclosed having rapid disintegration and low friability. In one embodiment, the disinfecting tablet comprises sodium dichloroisocyanurate dihydrate and at least one swellable disintegrating agent assisting with rapid disintegration of the tablet. Also disclosed are a disinfecting cleaning tool assembly and a method of cleaning.

### FIELD

The present invention relates to a disinfecting tablet. In particular, the present invention relates to a disinfecting tablet that disintegrates quickly but has low friability.

### BACKGROUND

Toilets may contain various microorganisms. Often, cleaning alone with detergents or mechanical abrasion is not sufficient to kill or inhibit the growth of these organisms and use of disinfectants is necessary. A disinfectant is a substance that destroys or irreversibly inactivates infectious or other undesirable bacteria, pathogenic fungi, and viruses on surfaces, or inanimate objects or in solutions.

Halogen releasing compounds are one example of an effective disinfecting agent. One example of a halogen releasing compound is chlorine-releasing agents. Chlorine-releasing agents that give off available chlorine when placed in water have been used as disinfectants to kill bacteria and to prevent their growth in such applications as bathroom and toilet cleaning, swimming pools, portable water supplies and the like. Chlorination has been accomplished by a variety of means using liquid solutions of chlorine-releasing agents or solid forms of agents which release chlorine.

Liquid disinfectant cleaners, such as liquid bleach, are commonly used. However, many liquid cleaners utilize high concentrations of corrosive chemicals, which easily spill or splatter on users causing chemical burns, inhalation burns, blindness or discomfort. Additionally, users must determine the appropriate amount to use for an application, which may be difficult resulting in using too little to accomplish proper disinfecting or using too much and wasting solution.

Another option is use of solid forms of disinfectants. Solid forms of disinfectants typically allow for higher concentrations of the active ingredient. In liquid form other ingredients may be present that interact and inactivate the active ingredients during storage, while solid forms are less likely to have this problem.

Solid forms of disinfectants may be in either granular or tablet form. Granular materials present the same problem as liquid cleaners in that they require the user to measure and therefore have the problem of spillage and storage of bulky materials. Also, granular materials of disinfectant, and particularly halogen compounds, may produce airborne particles that are breathed in by those handling the material such as the manufacturer, distributor, or user. These particles could cause damage to the person's mucous membranes or lung tissue.

A tablet disinfectant is easier to dose and handle and, due to its compact structure, is easier to store and transport. However, one problem that arises in the use of tablet disinfectants is the inadequate disintegrating rate of the tablets under use conditions. To make the tablets stable for packaging, transport, and handling, the tablets must be hard and heavily compacted. However, typically, such compact structures have slow disintegration rates in the liquid solution they are placed in to disinfect.

Disintegration aids can be included within the tablet to shorten the disintegration times. Disintegrants are substances added to the shaped bodies to accelerate their disintegration on contact with water. Generally, disintegration aids can be classified into one of three groups according to their mechanism of action. The first group is substances which increase the porosity or capillarity (“wick effect”) of tablets and which have a high adsorption capacity for water. These are referred to as swellable disintegrators. The second groups are gas-evolving substances for effervescent tablets. The third group is hydrophilicizing agents which ensure that the constituent particles of tablets are wetted in water.

The first class includes the substances known as traditional disintegrating agents, such as starch, cellulose and cellulose derivatives, alginates, dextrans, crosslinked polyvinyl pyrrolidones and many others. The second class includes systems of weak acids and carbonate-containing agents, more particularly citric acid and tartaric acid in combination with hydrogen carbonate or carbonate. Examples of the third class are polyethylene glycol sorbitan fatty acid esters.

However, even with the use of disintegrating agents such tablets may still be friable meaning that even though they are hard, upon handling they are brittle, easily break, and easily produce dust. As discussed, the dust particles may be breathed in by those handling the material and cause damage to their tissue. Also, easily breakable tablets present problems for transport, shipping and handling. Therefore, there remains a need for a fast disintegrating, disinfecting tablet with low friability.

### SUMMARY

The present invention relates to a disinfecting tablet. In particular, the present invention relates to a disinfecting tablet that disintegrates quickly but has low friability. The disinfecting tablet can be used to disinfect a liquid solution or can be used in combination with a cleaning tool to also clean the fixture holding the liquid solution.

In one embodiment, the disinfecting tablet comprises sodium dichloroisocyanurate dihydrate and at least one swellable disintegrating agent assisting with rapid disintegration of the tablet.

In one embodiment, the disinfecting tablet comprises a halogen releasing disinfecting agent and a swellable disintegrating agent. The swellable disintegrating agent comprises at least 10 wt % but less than 50 wt % of the tablet.

In one embodiment, the disinfecting tablet comprises a halogen releasing disinfecting agent and a disintegrating agent. The tablet has a disintegrating time of less than 20 seconds according to the Disintegration Test and a weight loss of less than 5% according to the Friability Test.

Also disclosed are a disinfecting cleaning tool assembly and a method of cleaning. In one embodiment a disinfecting cleaning tool assembly comprises a cleaning tool including a handle having a holding end and an operating end, a cleaning head including a slot for receiving the operating end of the cleaning tool, a disinfecting tablet embedded in the slot of the cleaning head. The cleaning head is attached to the cleaning tool and exposed to water such that the disinfecting tablet disintegrates.

In one embodiment, the method comprises providing a cleaning tool including a holding end and an operating end, providing a cleaning head including a slot for receiving the operating end of the cleaning tool, inserting a disinfecting tablet into the slot of the cleaning head, inserting the operating end of the cleaning tool into the slot, exposing the cleaning head to water, and disintegrating the disinfecting table in water.

### BRIEF DESCRIPTION OF THE DRAWINGS

FIG. 1 is a perspective view of a disinfecting tablet according to the present invention.

FIG. 2 is a perspective view of a disinfecting cleaning tool assembly;

FIG. 3 is a side sectional view of the disinfecting cleaning tool assembly of FIG. 2;

FIG. 4 is a perspective view of a cleaning pad for use with the disinfecting cleaning tool assembly of FIG. 2.

While the above-identified drawings and figures set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of this invention. The figures may not be drawn to scale.

### DETAILED DESCRIPTION

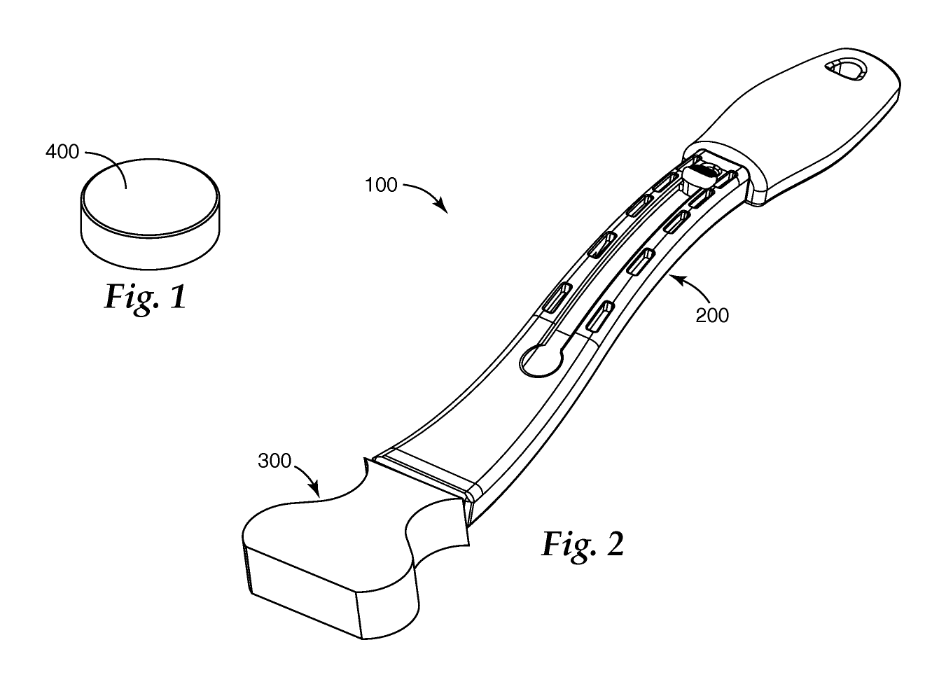

FIG. 1 is a perspective view of a disinfecting tablet 400 according to the present invention. In one embodiment, the disinfecting tablet includes a disinfecting agent and a disintegrating agent to aid in rapidly disintegrating the tablet in a liquid solution. In one embodiment, the disinfecting agent is a halogen releasing agent. Generally, the tablet contains at least 50% weight of the halogen releasing agent and at most 50% disintegrating agent. In one embodiment, the halogen releasing agent is a chlorine releasing agent. One example of a chlorine releasing agent is sodium dichloroisocyanurate dihydrate.

Other halogen releasing agents include alkali metal salts of dihalo cyanurates such as anhydrous sodium dichloroisocyanurate (NaDCCA), trichloroisocyanuric acid, calcium hypochlorite, alkali metal or alkaline earth metal hypochlorites such as lithium hypochlorite and calcium hypochlorite, hypochlorite addition products, chloramines, chlorimines, chloramides, chlorimides, heterocyclic N-Bromo and N-Chloro cyanurates, halogenated hydantoins, halogenated melamines, and inorganic hypohalite releasing agents.

The disintegrating agent aids in disintegrating the tablet. In one embodiment, the disintegrating agent is generally described as a swellable disintegrating agent. A swellable disintegrating agent is a disintegrating agent that absorbs the surrounding liquid. The absorption of the liquid causes the volume to increase, which causes the tablet to lose structural integrity and break apart. Generally, the tablet contains less than 50% weight of the disintegrating agent. In one embodiment, the disintegrating agent is a cellulose based agent. In one embodiment, the disintegrating agent also serves as a binder holding the particles of the disinfecting agent and the disintegrating agent together. One example of a swellable disintegrating agent is a combination of sodium carboxymethylcellulose and microcrystalline cellulose.

Other disintegrating agents includes starches such as sodium carboxymethyl starch or sodium-starch-glycolate, prepasted corn starch; cellulose and cellulose derivatives such as sodium carboxymethyl cellulose, microcrystalline cellulose; alginates; dextrans; crosslinked polyvinyl pyrrolidones; weak acids such as citric acid, sodium bisulfate, oxalic acid, malonic acid, succinic acid, glutaric acid, adipid acid, suberic acid, maleic acid, fumaric acid, sodium citrate, sodium sulfite, potassium citrate along with alkali carbonate-containing agents such as sodium bicarbonate, sodium carbonate, calcinated sodium carbonate, sodium sesquicarbonate, potassium bicarbonate and potassium carbonate.

Suitable optional additives to the tablet may include fillers, surfactants, perfumes, dyes, abrasives, binders and an effervescent compound. Stabilizing agents may be used for ensuring storage stability and activity under extreme environmental conditions. In one embodiment, the tablet may have various layers or coating of such things as, but not limited to, surfactants, abrasives or an effervescent compound.

The particular combination of disinfecting agent, disintegrating agent, and other ingredients, if included, is dependent on the compatibility of the compounds with the particular disinfecting agent. It is desirable to maintain the activity of the disinfecting agent during storage and aging. Also, it is desirable that the ability of the tablet to disintegrate quickly be maintained following storage.

Typically, the tablet contains at least 50 wt % of the disinfecting agent. In one embodiment, the tablet contains 50 to 90 wt % of the disinfecting agent. Typically, the tablet contains at most 50 wt % of the disintegrating agent. In one embodiment the tablet contains 10 to 50 wt % disintegrating agent.

One suitable disinfecting agent is sodium dichloroisocyanurate dihydrate. One suitable combination of disintegrating agents is microcrystalline cellulose and sodium carboxymethylcellulose. In this formulation the microcrystalline cellulose is serving as both a binder and a disintegrant.

In one embodiment, the tablet comprises 50 to 90 wt % sodium dichloroisocyanurate dihyrate, 10 to 40 wt % microcrystalline cellulose, and 0 to 5 wt % sodium carboxymethylcellulose. In one embodiment, the tablet comprises 70 to 75 wt % sodium dichloroisocyanurate dihydrate, 20 to 30 wt % microcrystalline cellulose, and 1 to 4 wt % sodium carboxymethylcellulose.

Sodium dichloroisocyanurate dihydrate is commercially available as CDB® 56 Medium from Clearon of South Charleston. W. Va. Microcrystalline cellulose is commercially available as Endurance™ MCC VE050 from FMC Corporation of Philadelphia, Pa. and has an average particle size of approximately 50 microns. Sodium carboxymethylcellulose is commercially available as Nymcel® ZSB-10 from C.P. Kelco of Atlanta, Ga. and is available as a fine powder and typically 10% remains on a standard 200 mesh screen or 90% through 200 mesh screen, which corresponds generally to a particles size of less than 74 microns.

To make a tablet, the disinfectant agent and disintegrant, and other compounds if included, are dry blended and then pressed to form a tablet. In one embodiment, sodium dichloroisocyanurate dihydrate, microcrystalline cellulose, and sodium carboxymethylcellulose are dry blended and pressed to form a tablet. In one embodiment, the tablets are pressed to an average hardness of at least 11 kP (as measured on a VanKel VK 200, model 40-2000). In one embodiment, the tablets are pressed to an average hardness of at least 18.5 kP. The tablets have a low friability and rapid disintegration in water.

Friable means readily crumbled or brittle and friability means the extent to which something is crumbled or brittle. The Friability Test includes placing 10 tablets each weighting approximately 1.38 grams and having a minimum hardness of approximately 18.5 kP in a Friabilator (previously available from VanKel Industries, Inc.). The Friabilator was tumbled for 100 revolutions at a rate of 25 revolutions per minute. The weight loss of the tablets was measured, and a cracked, cleaved or broken tablet is considered a failure. “Low friability” was determined to be having a weight loss of less than 5%, and at least 9 tablets that are not cracked, cleaved, or broken tablets. In one embodiment, “low friability” was determined to be having a weight loss of less than 1%, and no cracked, cleaved, or broken tablets.

Disintegrate means to separate into parts, to break up, or to deteriorate. Disintegration means the extent to which something will separate into parts, break up, or deteriorate. The Disintegration Test included placing approximately a 1.38 gram disinfecting tablet having a minimum hardness of 18.5 kP in 2250 mils of water with mild agitation. The disintegration time was measured from placement within the water until the tablet is visually dispersed in the liquid. “Rapid disintegration” was determined to be disintegrating in less than 20 seconds. In one embodiment, “rapid disintegration” was determined to be disintegrating in less than 15 seconds, and in one embodiment, “rapid disintegration” was determined to be disintegrating in less than 5 seconds.

The tablets may be in any shape or size such as, but not limited to, circular, oval, rectangular and may have generally curved or flat opposing major surfaces. In one embodiment, the tablet is circular with generally flat opposing major surfaces with a beveled edge adjacent the edge and the major surfaces. Flat opposing major surface are typically less likely to break, cleave, or crack during when pressed as compared to convex major surfaces. In one embodiment, the tablets range in weight from 0.75 grams to 5 grams. In the embodiment of a circular tablet, the tablet may have a diameter from 0.25 inches (6.35 mm) to 3 inches (7.62 cm) and a thickness from 0.079 inches (2.0 mm) to 0.276 inches (7.0 mm).

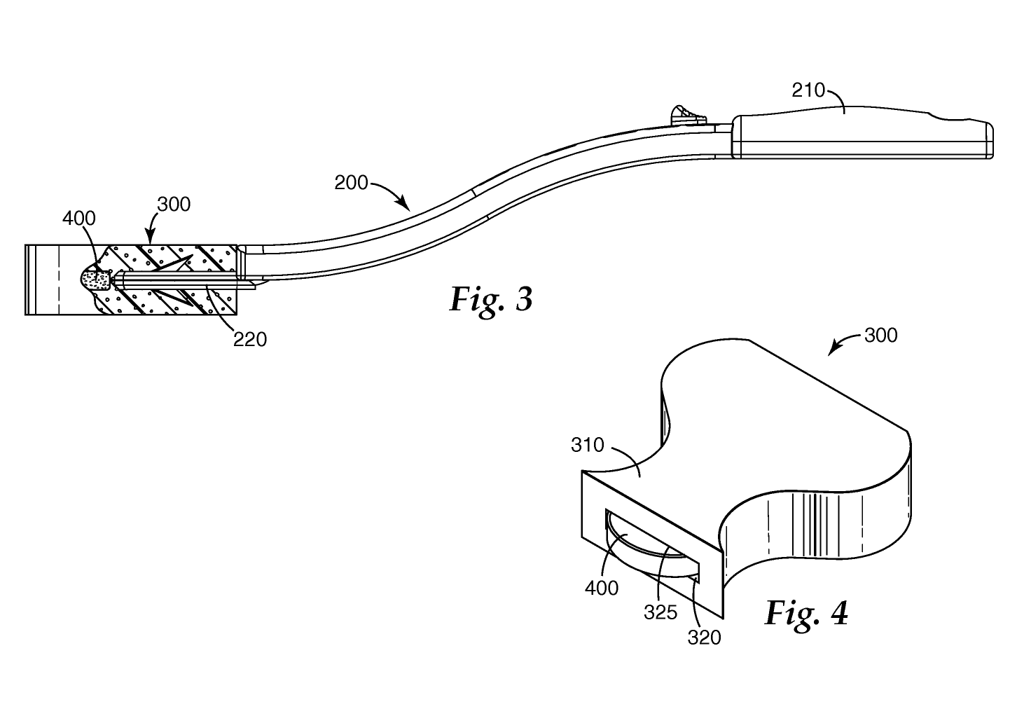

FIGS. 2 and 3 show a disinfecting cleaning tool assembly 100. The cleaning tool assembly includes a cleaning handle 200 and a cleaning pad 300 embedded with a disinfecting tablet 400. The cleaning handle 200 includes a holding end 210 (FIG. 3) and an operating end 220 (FIG. 3), opposite the holding end 210. The holding end 210 is designed for a user to grip, hold, and manipulate the cleaning handle 200. The operating end 220 is designed to receive and retain the cleaning pad 300 and perform the cleaning function. The operating end 220 may include a means for retaining the cleaning pad 300 during use and a means for ejecting a used cleaning pad 300. The cleaning handle 200 and cleaning pad 300, including a retention and ejecting mechanism, may be similar to that described in U.S. Pat. No. 7,146,676 filed on Sep. 16, 2003 and issued on Dec. 12, 2006, the disclosure of which is herein incorporated by reference.

FIG. 3 is a side sectional view of the cleaning tool assembly 100 of FIG. 1. Embedded within the cleaning pad 300 is a disinfecting tablet 400, such as described above. As shown in FIG. 3, the disinfecting tablet 400 is contained within the cleaning pad 300 and when the operating end 220 is also inserted in the slot 320, the operating end 220 of the cleaning handle 200 prevents the disinfecting tablet 400 from becoming dislodged.

FIG. 4 is a perspective view of the cleaning pad 300 for use with the disinfecting cleaning tool assembly of FIG. 2 showing the disinfecting tablet 400 being inserted into an opening 325 in the cleaning pad 300. It is understood that prior to insertion of the operating end 220 of the cleaning handle 200, the disinfecting tablet 400 would be entirely embedded within the cleaning pad 300 and would not be visible.

In one embodiment, the cleaning pad 300 is a pad of stiff resiliently flexible fibrous material (e.g., preferably a pad of non-woven polymeric fibers such as the material sold under the trade designation “Scotchbrite” by 3M Company, St. Paul, Minn., although other materials could be used), to which fibers may be adhered an abrasive which helps to clean but will not scratch toilet bowls, (e.g., the abrasive mineral commercially designated “Minex” that is available from Cary Co., Adison, Ill.). The cleaning pad 300 may, optionally, be impregnated with all or any one or ones of a cleaning material (e.g., detergent), a dye, and a fragrance. Additionally, the cleaning pad 300 may include abrasive particles releasably secured to the cleaning pad 300 by a water-soluble binder or surfactant such as described in U.S. patent application Ser. No. 11/051,838, filed on Feb. 4, 2005, the disclosure of which is herein incorporated by reference.

The cleaning pad 300 shown has opposite parallel generally omega shaped major surfaces 310 and has contacting or closely spaced inner surfaces midway between and parallel to its major surfaces 310 that define a slot 320 with an opening 325 through one edge surface of the cleaning pad 300. The disinfecting tablet 400 is inserted into the opening 325 and embedded within the slot 320 of the cleaning pad 300. When the operating end 220 of the cleaning handle 200 is inserted into opening 325 and retained in slot 320, the tablet 400 remains securely engaged within the slot 320 and is unable to become dislodged. However, due to the porosity of the cleaning pad 300, the disinfecting agent is able to be dispersed out of the cleaning pad 300 and onto the surface to be cleaned or into the solution being cleaned. The construction of the cleaning pad 300 also provides a cushioning effect to prevent breaking or damage during shipping or handling to the embedded disinfecting tablet 400.

It is understood that any type of cleaning tool and cleaning pad may be used in combination with the disinfecting tablet described. For example, the cleaning tool may or may not have an operating end with a retaining mechanism or an ejecting mechanism.

If included, the cleaning pad may be constructed from a variety of materials including, but not limited to, a lofty web of fibers forming a nonwoven, and sponge or foam. Additionally, the cleaning pad may be formed from suitable paper-like components that disintegrate in water such that the cleaning pad is flushable.

As shown, the disinfecting tablet 400 is securely retained within the slot 320 due to the frictional effect of the interaction with the fibers of the web. A separate attachment mechanism may be included to secure the disinfecting tablet 400 within the cleaning pad 300. For example, needle punching around the perimeter of the tablet or use of an adhesive may be used. It is understood that even with inclusion of a cleaning pad, the disinfecting tablet 400 does not necessarily need to be embedded within the cleaning pad 300. The disinfecting tablet 400 may be placed on the exterior of the cleaning pad 300. Even if embedded within the cleaning pad 300, the disinfecting tablet 400 does not necessarily need to be embedded in a slot. Other types of openings, pouches, or holding mechanisms can be included for supporting the disinfecting tablet 400.

It is understood that the cleaning tool assembly shown is primarily for use in cleaning a toilet. However, a cleaning tool assembly with a cleaning tool and cleaning pad having a disinfecting tablet may be used for cleaning other surfaces such as but not limited to floors, showers, or tubs.

Although specific embodiments of this invention have been shown and described herein, it is understood that these embodiments are merely illustrative of the many possible specific arrangements that can be devised in application of the principles of the invention. Numerous and varied other arrangements can be devised in accordance with these principles by those of ordinary skill in the art without departing from the spirit and scope of the invention. Thus, the scope of the present invention should not be limited to the structures described in this application, but only by the structures described by the language of the claims and the equivalents of those structures.

### EXAMPLES

### Test Methods

### Tablet Hardness

Tablet hardness was measured using a VK 200 Tablet Hardness Tester, Model 40-2000, previously available from VanKel Industries, Inc. A VK 200 Tablet Hardness Tester is currently available from Varian, Inc., Palo Alto, Calif. The hardness data is reported in units of kP.

### Tablet Disintegration Time

To measure disintegration time, the tablet was placed in a beaker of water containing about 2250 milliliters of cold tap water with mild agitation using a magnetic stirrer bar. The tablet was swirled around the edge of the beaker with the magnetic stirrer bar never contacting the tablet. The time that the tablet took to disintegrate was recorded. Initial tablet disintegration time was measured and in some cases the disintegration time was measured for 3- or 6-day old tablets that had been stored at ambient or room temperature (around 65° F.).

### Tablet Friability

The friability test replicates the conditions that the tablet may be subjected to during various phases of manufacturing, distribution and handling. Friability testing was carried out using a Friability Tester previously available from VanKel Industries, Inc. A similar Friability Tester is currently available from Varian, Inc., Palo Alto, Calif.

Ten tablets were tested for each example. Any loose dust was first removed from the tablets. The tablets were then accurately weighed. The tablets were then placed in the drum of the tester and were tumbled for 100 revolutions at a rate of 25 revolutions per minute (RPM). The tablets were removed from the drum and any loose dust was removed. The tablets were reweighed and the percent weight loss was calculated. If more than one tablet present in the tablet sample was obviously cracked, cleaved or broken after tumbling, the sample failed the test. Initial tablet friability was tested and in some cases the friability was tested for 3-day old tablets that had been stored at ambient or room temperature (around 65° F.). The number of tablets remaining intact after tumbling and/or the percent weight loss was recorded.

### Materials

CBD® 56 Medium: Sodium dichloroisocyanurate dihydrate, available from Clearon of South Charleston, W. Va.
Endurance™ MCC VE-050: Microcrystalline cellulose having an average particle size of approximately 50 microns, available from FMC Corporation of Philadelphia, Pa.
Avicel® PH-102: Microcrystalline cellulose having an average particle size of approximately 50 microns, available from FMC Corporation of Philadelphia, Pa.
Prosolv® HD 90: Silicified high density microcrystalline cellulose composed of 98% microcrystalline cellulose and 2% colloidal silicon dioxide, available from JRS Pharma LP, Patterson, N.Y.
Nymcel® ZSB-10: Sodium carboxymethylcellulose, available from C.P. Kelco of Atlanta, Ga.
Disintex 75: Polyvinylpolypyrrolidone, 140 mm particle size, available from International Specialty Products, Wayne, N.J.
Vivasol®: Croscarmallose Sodium, available from JRS Pharma LP, Patterson, N.Y.
Accelerate™: Croscarmallose Sodium, available from FMC Corporation of Philadelphia, Pa.
Sodium Biocarbonate

### EXAMPLES

The disinfecting tablet compositions are summarized in Table 1. The amounts given are in weight percent solids. The integredients were mixed and subsequently compressed using a conventional tablet press into circular shaped tablets having either a flat or convex surface profile. In general, for a tablet approximately 1.38 g the average diameter was 15.9 mm and the thickness was 4.9 mm. The tablet weight, shape and hardness are provided in Table 1.

TABLE 1

| | CDB ® | Endurance ™ | | | | | | | | Tablet | | Tablet |
|---|---|---|---|---|---|---|---|---|---|---|---|---|
| | 56 | MCC | Avicel | Prosolv ® | Disintex | Nymcel ® | | | Sodium | Weight | Tablet | Hardness |
| Ex. | Medium | VE-050 | PH102 | HD 90 | 75 | ZSB-10 | Vivasol ® | Accelerate ™ | bicarbonate | (grams) | profile | (kP) |
| 1 | 77.5 | — | 20 | — | 2.5 | — | — | — | — | 1.32 | convex | 12.5 |
| 2 | 77.5 | 20 | — | — | 2.5 | — | — | — | — | 1.29 | flat | 14.2 |
| 3 | 77.5 | 20 | — | — | — | — | 2.5 | — | — | 1.34 | flat | 21.1 |
| 4 | 77.5 | — | 20 | — | 2.5 | — | — | — | — | 1.28 | flat | 16.1 |
| 5 | 72.5 | 25 | — | — | 2.5 | — | — | — | — | 1.36 | flat | 14.4 |
| 6 | 72.5 | 25 | — | — | — | 2.5 | — | — | — | 1.37 | flat | 19.1 |
| 7 | 85 | — | — | 15 | — | — | — | — | — | 1.28 | convex | 11.3 |
| 8 | 87.5 | — | 10 | — | — | 2.5 | — | — | — | 1.29 | convex | 13.9 |
| 9 | 87.5 | — | 10 | — | — | — | 2.5 | — | — | 1.28 | convex | 12.4 |
| 10 | 87.5 | — | 10 | — | — | — | | 2.5 | — | 1.27 | convex | 15.4 |
| 11 | 65 | 20 | — | — | — | — | — | — | 15 | 1.52 | flat | 14.6 |
| 12 | 65 | 20 | — | — | — | — | — | — | 15 | 1.54 | flat | 20.7 |
| 13 | 60 | 20 | — | — | — | — | — | — | 20 | 1.71 | flat | 12.3 |
| 14 | 60 | 20 | — | — | — | — | — | — | 20 | 1.70 | flat | 17.4 |

The resultant tablets were tested for disintegration time and friability according to the above test methods. Results are provided in Table 2.

TABLE 2

| | | | | | 3 Day Old |
|---|---|---|---|---|---|
| | | 3 Day Old | 6 Day Old | Initial | Tablet |
| | | Tablet | Tablet | Friability | Friability |
| | Initial Tablet | Disintegration | Disintegration | (number of | (number of |
| | Disintegration | Time | Time | unbroken | unbroken; % |
| Example | Time (seconds) | (seconds) | (seconds) | tablets) | weight loss) |
| 1 | 3 | — | 21.0 | 10 | — |
| 2 | 3 | 7.5 | — | — | 9 |
| 3 | 12 | 15.0 | — | — | 10; 0.30 |
| 4 | 3 | 11.0 | — | — | 9 |
| 5 | 3 | 9.5 | — | — | 10; 1.60 |
| 6 | 6 | 6.5 | — | — | 10; 0.62 |
| 7 | 3 | — | 43.0 | 9 | — |
| 8 | 9 | — | 28.0 | 10 | — |
| 9 | 5 | — | 40.0 | 10 | — |
| 10 | 16 | — | 110.0 | 10 | — |
| 11 | 6 | 6.5 | — | — | 10; 1.40 |
| 12 | 10 | 9.0 | — | — | 10; 0.43 |
| 13 | 4 | 5.5 | — | — | 10; 1.00 |
| 14 | 5 | 7.0 | — | — | 10; 0.40 |

What is claimed is:

1. A disinfecting tablet comprising:
sodium dichloroisocyanurate dihydrate; and
at least one swellable disintegrating agent assisting with rapid disintegration of the tablet.

2. The disinfecting tablet of claim 1, wherein the tablet comprises at least 50 wt % sodium dichloroisocyanurate dihyrate.

3. The disinfecting tablet of claim 1, further including a binder.

4. The disinfecting tablet of claim 1, wherein the swellable disintegrating agent is also a binder.

5. The disinfecting tablet of claim 1, where in the swellable disintegrating agent is cellulose product.

6. The disinfecting tablet of claim 5, wherein the swellable disintegrating agent is sodium carboxymethylcellulose.

7. The disinfecting tablet of claim 5, wherein the swellable disintegrating agent is microcrystalline cellulose.

8. The disinfectant tablet of claim 1, comprising 50 to 90 wt % sodium dichloroisocyanurate dihydrate, 10 to 40 wt % microcrystalline cellulose, and 0.5 to 5 wt % sodium carboxymethylcellulose.

9. The disinfecting tablet of claim 1, comprising 72.5 wt % sodium dichloroisocyanurate dihydrate, 25 wt % microcrystalline cellulose, and 2.5 wt % sodium carboxymethylcellulose.

10. The disinfecting tablet of claim 1, wherein the tablet including a hardness of at least 18 kP.

11. A disinfecting tablet comprising:
a halogen releasing disinfecting agent;
a swellable disintegrating agent;
wherein the swellable disintegrating agent comprises at least 10 wt % but less than 50 wt % of the tablet.

12. A disinfecting tablet comprising:
a halogen releasing disinfecting agent and a disintegrating agent, wherein the tablet has a disintegrating time of less than 20 seconds according to the Disintegration Test and a weight loss of less than 5% according to the Friability Test.

13. A disinfecting cleaning tool assembly comprising:
a cleaning tool including a handle having a holding end and an operating end;
a cleaning head including a slot for receiving the operating end of the cleaning tool;
a disinfecting tablet embedded in the slot of the cleaning head;
wherein the cleaning head is attached to the cleaning tool and exposed to water such that the disinfecting tablet disintegrates.

14. The disinfecting cleaning tool assembly of claim 13, wherein the cleaning head is a lofty web of fibers.

15. The disinfecting cleaning tool assembly of claim 13, wherein the disinfecting tablet includes a halogen releasing disinfecting agent and a disintegrating agent.

16. A method of cleaning comprising:
providing a cleaning tool including a holding end and an operating end;
providing a cleaning head including a slot for receiving the operating end of the cleaning tool;
inserting a disinfecting tablet into the slot of the cleaning head;
inserting the operating end of the cleaning tool into the slot;
exposing the cleaning head to water;
disintegrating the disinfecting tablet in water.

17. The method of cleaning of claim 16, further cleaning a surface with the cleaning head.

18. The method of cleaning of claim 16, further comprising discarding the cleaning head.

19. The method of cleaning of claim 16, wherein the disinfecting tablet disintegrates in less than 20 seconds according to the Disintegrating Test.

20. The method of cleaning of claim 16, wherein the disinfecting tablet has a weight loss of less than 5% according to the Friability Test.
